(19) United States
(12) Patent Application Publication
(10) Pub. No.: US 2003/0134893 A1
(43) Pub. Date: Jul. 17, 2003

(54) Combination chemotherapy

(76) Inventors: Wayne Daniel Klohs, Ypsilanti, MI (US); Ronald Lynn Merriman, Ann Arbor, MI (US)

Correspondence Address: Steven R. Eck, Warner-Lambert Company, 2800 Plymouth Road, Ann Arbor, MI, 48105, US

(21) Appl. No.: 10/286,498

(22) Filed: Feb. 20, 2003

Related U.S. Application Data
(62) Division of application No. 10/148,063, filed on May 24, 2002.
Application No. 10/148,063 is a 371 of international application No. PCT/US00/30377, filed on Nov. 3, 2000.
(60) Provisional application No. 60/164,889, filed on Nov. 10, 1999.
(60) Provisional application No. 60/222,561, filed on Aug. 3, 2000.

(51) Int. Cl. A61K 31/337; A61K 31/16

(52) U.S. Cl. 514/449; 514/616

(57) ABSTRACT

Acetyldinaline in combination with paclitaxel and/or carboplatin is synergistic for treating cancer.

### FIELD OF THE INVENTION

The invention concerns a method for treating tumors utilizing a combination of known oncolytic agents. The use of the agents together provides unexpectedly greater efficacy than employing the single agents alone.

### BACKGROUND OF THE INVENTION

Cancer chemotherapy has advanced dramatically in recent years. Many tumors can be effectively treated utilizing compounds which are either naturally occurring products or synthetic agents. Cancer chemotherapy often entails use of a combination of agents, generally as a means of providing greater therapeutic effects and reducing the toxic effects that are often encountered with the individual agents when used alone.

We have now discovered a unique combination of known oncolytic agents which exhibits a dramatic synergistic effect. The combination utilizes the agent acetyldinaline, together with paclitaxel and/or carboplatin. The combination is especially effective in treating patients with solid tumors, especially nonsmall cell lung cancer and other advanced solid tumors.

Acetyldinaline is 4-acetylamino-N-(2′-aminophenyl)-benzamide. It is also known as CI-994. It is described in U.S. Pat. No. 5,137,918, which is incorporated herein by reference for its teaching of how to make acetyldinaline, how to formulate it into dosage forms, and how to use it for treating cancers such as colon cancer and adenocarcinomas. Acetyldinaline is also described in U.S. Pat. No. 5,795,909 as a possible conjugate for cancer treatment.

Paclitaxel is a natural product mitotic inhibitor. It is an antimicrotubule agent that promotes the assembly of microtubules from tubulin dimers and stabilizes microtubules by preventing depolymerization. This stability results in the inhibition of the normal dynamic reorganization of the microtubule network that is essential for vital interphase and mitotic cellular functions. In addition, paclitaxel induces abnormal arrays or bundles of microtubules throughout the cell cycle and multiple asters of microtubules during mitosis. Paclitaxel is indicated primarily for ovarian carcinoma and breast cancer, although it is useful in treating other cancers as well. Use of paclitaxel is generally accompanied by undesirable side effects, including hypersensitivity reactions, hypotension, bradycardia, hypertension, nausea and vomiting, and injection site reactions. Paclitaxel is commercially available as Taxol® (Bristol-Myers Squibb).

Carboplatin is a bicyclic compound having platinum as a ring atom. The compound is routinely used clinically as a cancer chemotherapeutic agent, especially for ovarian carcinoma, as well as nonsmall cell lung cancer (NSCLC). Carboplatin generally is used in combination with other antineoplastic agents, and the combination use of carboplatin and paclitaxel has become widely used for treating patients with advanced NSCLC, ovarian cancer, and other serious cancers (see Calvert et al., J. Clin. Oncol., 1989;7:1748-1756). Carboplatin is available commercially as Paraplatin® (Bristol-Myers Squibb). The product is supplied as a crystalline white powder in vials containing 50, 150, and 450 mg, and the powder is reconstituted with either Sterile Water for Injection, 5% Dextrose in Water, or 0.9% Sodium Chloride for Injection

An object of this invention is to provide a method for treating cancers, especially advanced solid tumors, with a combination comprising acetyldinaline together with paclitaxel or carboplatin, or a combination comprising acetyldinaline, paclitaxel, and carboplatin. A further object is to provide a composition comprising synergistic amounts of acetyldinaline and paclitaxel, acetyldinaline and carboplatin, and acetyldinaline and both paclitaxel and carboplatin.

### SUMMARY OF THE INVENTION

This invention relates to a synergistic combination of antineoplastic agents, and to a method for treating tumors comprising administering the combination. The invention more particularly provides a composition comprising, as a first component, acetyldinaline, and as a second component, paclitaxel or carboplatin. The invention also provides a composition comprising all three agents together.

The compositions of this invention consist essentially of the above active ingredients, or suitable salts thereof, together with common excipients, diluents, and carriers.

In a further embodiment of the invention, we provide a method for treating cancer comprising administering to an animal in need of treatment an effective amount of a combination of acetyldinaline with either paclitaxel or carboplatin, or a combination of acetyldinaline, paclitaxel, and carboplatin.

A preferred method embraces treatment of solid tumors.

A further preferred method employs an antitumor amount of acetyldinaline and an effective amount of paclitaxel to treat susceptible cancers, including NSCLC, breast cancer, ovarian cancer, head and neck cancer, myelomas, prostate cancer, and pancreatic cancer.

A further preferred method employs an antitumor amount of acetyldinaline and an effective amount of carboplatin to treat susceptible cancers.

A further preferred method employs an antitumor amount of acetyldinaline plus an effective amount of paclitaxel plus an effective amount of carboplatin to treat susceptible cancers.

Another embodiment of the invention is a kit comprising in one compartment a dosage of acetyldinaline, and in another compartment a dosage of paclitaxel.

Another embodiment of the invention is a kit comprising in one compartment a dosage of acetyldinaline, and in another compartment a dosage of carboplatin.

Another embodiment of the invention is a kit comprising in one compartment a dosage of acetyldinaline, and in another compartment a dosage of paclitaxel, and in a third compartment a dosage of carboplatin.

### DESCRIPTION OF FIGURES

FIG. 1 shows the synergy of CI-994 and paclitaxel in mouse colon cancer cells.

FIG. 2 shows the anti-tumor activities of CI-994, paclitaxel and carboplatin, each alone, and the combination treatment with all three.

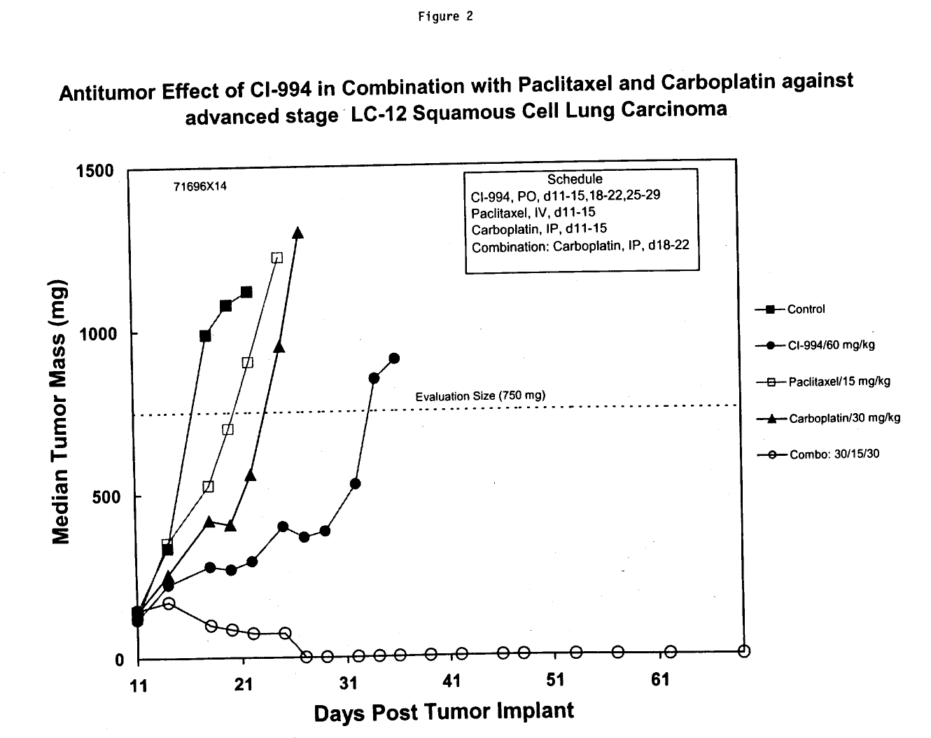

### DETAILED DESCRIPTION OF THE INVENTION

The compounds to be utilized in the method of this invention will be administered in doses commonly employed clinically. Such doses will be calculated in the normal fashion, for example on body surface area. Acetyldinaline will be administered, for example, at doses from about 1.0 mg/m² to about 50 mg/m², preferably from about 2.0 mg/m² to about 10.0 mg/m². Ideally, acetyldinaline will be administered at a dose which will produce plasma levels of about 5 to about 100 μg/mL. Acetyldinaline typically is administered orally, for example, as capsules having active ingredient in the amounts of 2.5, 5, and 25 mg per capsule. Acetyldinaline will be administered daily at about the same dose levels throughout a treatment period, typically for 15 to 30 days. Multiple treatment periods can be practiced, as dictated by the attending medical practitioner and the particular patient and condition being treated.

Paclitaxel is a cytotoxic anticancer drug, and caution should be exercised in handling the agent. Paclitaxel typically is provided in vials, and is diluted prior to administration by intravenous infusion. Typical diluents include 0.9% sodium chloride, 5% dextrose. The final concentration for infusion generally is about 0.3 to about 1.2 mg/mL. Paclitaxel is commercially available in several concentrations, for instance, 30 mg/5 mL multidose vials, 100 mg/16.7 mL multidose vials, and 300 mg/50 mL multidose vials. The product generally is administered, for treatment of ovarian cancer for example, at doses of about 135 mg/m² to about 225 mg/m² over 3 hours every 3 weeks, generally in increments of about 25 mg/m².

The agent is an effective treatment for carcinoma of the breast at doses of 175 mg/m² administered IV over 3 hours every 3 weeks. For treatment of AIDS-related Kaposi's sarcoma, paclitaxel generally is given IV at 135 mg/m² over 3 hours every 3 weeks, or at 100 mg/m² over 3 hours every 2 weeks. In general, the dosage intensity of paclitaxel will be about 45 to 50 mg/m²/week.

Carboplatin is administered as an IV infusion over a period of about 30 minutes to about 1 hour. The dosage commonly used will be about 25 mg to about 500 mg for each treatment course, for example, each day for about 12 to 14 days, followed by about 4 to 6 days of no dosing, and then repeated dosing for another 12 to 14 days.

In a preferred embodiment, a typical invention combination will be administered at the following doses shown in Table 1.

TABLE 1

| Paclitaxel Dose (mg/m² | Acetyldinaline Dose (oral doses |
|---|---|
| infused over 3 hours on Day 1) | administered daily on Days 1-14) |
| 175 | 2.5 mg fixed dose |
| 175 | 5 mg fixed dose |
| 200 | 6 mg/m²/day |
| 225 | 4 mg/m²/day |

Another typical dosing embodiment is shown in Table 2.

TABLE 2

| Carboplatin Dose (mg | Acetyldinaline Dose (orally dosed |
|---|---|
| infused over 30 minutes on Day 1) | each day for Days 1-14) |
| 150 | 2.5 mg fixed dose |
| 200 | 6 mg fixed dose |
| 300 | 6 mg/m²/day |
| 500 | 4 mg/m²/day |

An especially preferred embodiment is the combination of all three agents, and typical dosing is shown in Table 3.

TABLE 3

| Paclitaxel Dose (mg/m²) | Carboplatin | Acetyldinaline (orally |
|---|---|---|
| (infused over 3 hours on | (infused over | dosed daily on |
| Day 1 | 30 minutes on Day 1) | Days 1-14) |
| 175 | 300 | 2.5 mg fixed dose |
| 175 | 400 | 5 mg fixed dose |
| 175 | 350 | 4 mg/m²/day |
| 200 | 450 | 6 mg/m²/day |
| 250 | 200 | 4 mg/m²/day |

The combinations provided by this invention have been evaluated in several assay systems, and the data can be analyzed utilizing a standard program for quantifying synergism, additivism, and antagonism among anticancer agents. The program preferably utilized is that described by Chou and Talalay, in “New Avenues in Developmental Cancer Chemotherapy,” Academic Press, 1987, Chapter 2.

The method is based on the median-effect principle of the mass-action law using an enzyme kinetic system as a model. The equation is simple and describes the relationships between dose and effect regardless of the shape of the dose-effect curve. Two basic equations constitute the pillars of this methodology. To relate dose and effect for a single drug in the simplest way possible, the median-effect equation derived by Chou is given by:

fₐ/fᵤ=(D/Dₘ)^{m}

D=Dₘ[fₐ/(1−fₐ)]^{1/m}

where the right side represents the dose and the left side represents the effect, in which fₐ and fᵤ are the fractions affected and unaffected, respectively, D is the dose, Dₘ is the median-effect dose signifying the potency, and m is a coefficient signifying the shape of the dose-effect curve. From this equation Chou and Talalay derived the general equation for two or more drugs:

$$\begin{matrix}{[ \frac{{( {f}_{a} )}_{1 , 2}}{{( {f}_{u} )}_{1 , 2}} ]}^{1 / m} = {[ \frac{{( {f}_{a} )}_{1}}{{( {f}_{u} )}_{1}} ]}^{1 / m} + {[ \frac{{( {f}_{a} )}_{2}}{{( {f}_{u} )}_{2}} ]}^{1 / m} = + {α [ \frac{{( {f}_{a} )}_{1} {( {f}_{a} )}_{2}}{{( {f}_{u} )}_{1} {( {f}_{u} )}_{2}} ]}^{1 / m} \\ = \frac{{( D )}_{1}}{{( {D}_{m} )}_{1}} + \frac{{( D )}_{2}}{{( {D}_{m} )}_{1}} = \frac{{α ( D )}_{1} {( D )}_{2}}{{( {D}_{m} )}_{1} {( {D}_{m} )}_{2}}\end{matrix}$$

where m=1 is for first-order Michaelis-Menten-type kinetics and m>1 (or m<1) is for higher order (or lower order) Hill-type kinetics. When alpha=0, the third term on the right side disappears and when alpha=1, the third term is conserved. Alpha=0 is used for mutually exclusive drugs and alpha=1 is used for mutually nonexclusive drugs. For drugs that have the same or similar modes of action, the effects of both drugs are mutually exclusive. For drugs that have different modes of action or act independently, the effects of both drugs are mutually nonexclusive.

A plot of fraction affected (Fₐ) versus combination index (CI) is called the Fₐ-CI plot. This plot indicates synergism, additivity, or antagonism of two drugs at various effect levels in a mixture that is serially diluted. If several mixtures are made, it is possible to estimate the optimal combination ratio for maximal synergy. Different effect levels usually give different degress of synergism, additivism, or antagonism. CI values <1 indicate synergism; CI values >1 indicate antagonism, and CI values that are one or hover around one indicate additivity. For anticancer agents, synergism at high effect levels (Fₐ) is clinically more relevant than synergism at low Fₐ levels.

While acetyldinaline (CI-994) has not been approved for clinical use, it has nevertheless been evaluated in several clinical trials. In one such study, patients were treated using a dose-escalation scheme that increased both the daily dose and the duration of treatment. The majority of patients had received extensive prior chemotherapy. The maximum-tolerated dose (MTD) was 15 mg/m²/day when the duration of treatment was 14 consecutive days. To allow more prolonged treatment, lower doses were studied. Using a schedule of 8 weeks of continuous daily therapy, followed by a 2-week ‘drug holiday’, the MTD was 8 mg/m²/day. The dose-limiting toxicity was thrombocytopenia or neutropenia, usually occurring within 1 month of the start of therapy. Blood counts tended to stabilize even with continued treatment and to recover quickly when treatment was stopped. There was no evidence of cumulative toxicity following repeated courses and prolonged exposures to CI-994. Other toxicities included nausea, vomiting, diarrhea, anorexia, fatigue, mucositis, headache, dehydration, and increases in liver and renal function test values. Responses included one partial response in a heavily pretreated patient with NSCLC and a minor response in one patient each with renal cell cancer and NSCLC.

An additional Phase 1 study was conducted in patients with relapsed acute leukemia or other hematologic malignancy using a once daily high-dose 5-day dosing schedule. The MTD was 135 mg/m²/day. The dose-limiting toxicity was acute CNS toxicity manifested as sedation and somnolence. Other adverse events included nausea, vomiting, hypotension resulting from dehydration, hypocalcemia, headache, and in one patient each, acute pancreatitis, a pyramidal syndrome characterized by hyperreflexia and bilateral Babinski reflexes, and sepsis. Hematologic toxicities cannot be assessed in this patient population. Two patients with AML developed tumor lysis syndrome, resulting in one death. Transient decreases in peripheral white blood cell counts were noted.

A Phase 2 program is currently being conducted with CI-994, used as a single agent. The dosing regimen is 8 mg/m² given orally daily. Over 100 patients have been treated, including patients with nonsmall cell lung cancer, renal cell cancer, pancreatic cancer, head and neck cancer, ovarian cancer, myeloma, prostate cancer, and breast cancer. Some patients have tolerated dose increases to 10 mg/m², while some patients have had to have treatment interrupted due to thrombocytopenia, and then be restarted on CI-994 at lowered doses. The adverse events have been similar to those observed in the chronic dosing Phase 1 protocol. Thrombocytopenia has been the dose-limiting toxicity. Infrequent neurologic adverse events including paresthesias, confusion, and hallucinations have been reported. Objective responses have been seen in patients with nonsmall cell lung cancer. Clinical benefit has been reported in patients with renal cell cancer.

In the solid tumor Phase 1 study, CI-994 doses were administered orally following a fasting period, and blood samples were collected for pharmacokinetic analyses. Preliminary results indicate that the maximum blood level is achieved approximately 1 to 2 hours after ingestion, and the terminal elimination half-life of CI-994 is approximately 15 hours. The maximum plasma CI-994 concentrations achieved with increasing dose levels were less than dose-proportional. The terminal elimination half-life and the apparent clearance rate were independent of the dose administered.

One additional objective of this study was to determine whether taking CI-994 with food affected its rate or degree of absorption. Twelve fasted patients were given a single dose of CI-994, 8 mg/m². One week later, the same patients were given the same dose of CI-994 with a normal meal. Analysis of pharmacokinetic data revealed that CI-994 can be taken without regard to meals.

Mass balance/route of elimination studies have not been conducted in humans. Animal studies indicate that the principal route of elimination is via renal excretion, with 80% and 62% of radiolabeled drug appearing in the urine of monkeys and rats, respectively, within 24 hours.

The following detailed examples further establish the synergy between CI-994 and paclitaxel and/or carboplatin.

### EXAMPLE 1

The synergistic combinations provided by this invention have been evaluated in standard chemotherapy studies using female BALB/C mice weighing 18 to 20 g. The test mice were obtained from Charles River Laboratories, Wilmington, Mass. On Day 0 of the test, each mouse was surgically implanted (subcutaneously) with a fragment of LC-12 squamous cell lung carcinoma tumor weighing approximately 30 to 60 mg. The tumor fragments were implanted using a 12 gauge trocar. The mice were weighed weekly, and tumor size (width and length in mm) were measured two times each week with standard calipers. Tumor mass for each animal was calculated according to the formula:

$$Tumor weight ( mg ) = \frac{( a \times {b}^{2} )}{2} ,$$

where “a” is width of the tumor in mm, and “b” is the length in mm. Evaluation of anticancer activity was established by the formula T−C, where “T” and “C” are the median time (in days) required for the treated and control (respectively) tumors to reach a predetermined a size of about 750 mg (the “evaluation size”). Tumors generally reached size of about 150 to about 200 mg before drug dosing was initiated. Antitumor activity was assessed according to four parameters: (1) partial tumor response (PR); (2) complete tumor response (CR); (3) tumor-free survival (TF); and (4) tumor growth delay (TL). Tumor growth delay is expressed as a T−C value, where T is the median days required for the treatment group tumors to reach a predetermined size (e.g., 750 mg), and C is the median days for the control group tumors to reach this size. From the tumor growth delay value, the net log₁₀ tumor cell kill is calculated as follows:

Net log₁₀ tumor cell kill=[(T−C)−Rx]/3.32×Td

where Td is the days for the tumor mass to double, and Rx is the total days of treatment. Td is estimated from the best fit straight line from a log-linear plot of the control-group tumors in exponential growth (200 to 800 mg range). The conversion of the T−C values to log₁₀ cell kill is possible because the Td for tumors regrowing after treatment is approximately the same as that for untreated control mice. The net log₁₀ kill value normalizes the efficacy data for tumor growth during treatment regimens of varied duration and provides an estimate of whether an actual regression of the tumor occurred. Positive values indicate that an actual reduction of tumor burden occurred. Negative values indicate the tumor actually grew (although possibly more slowly) during treatment. Tumor-free survivors were excluded from these calculations.

Acetyldinaline was suspended in 0.5% aqueous methyl cellulose and administered orally at various dosages in 0.5 mL volumes. Taxol was dissolved in 0.1% aqueous saline and administered intravenously at various dosage levels in 0.5 mL injections.

The animals were divided into groups of eight animals each. One group served as controls and received no drug treatments. Four groups received oral doses of acetyldinaline alone at a specified level of active drug (7.5 mg/kg, 15 mg/kg, 30 mg/kg, and 60 mg/kg). The acetyldinaline was administered daily on Days 11-15 (Day 0 being when the tumor was implanted), Days 18-22, and Days 25-29. One group received Taxol alone at doses of 15 and 20 mg/kg. Four groups received acetyldinaline at the recited doses, in combination with 15 mg/kg of Taxol, and another four groups received acetyldinadine at the recited doses in combination with Taxol at 20 mg/kg.

### Results and Conclusion

The antitumor effects that are produced when CI-994 is used in combinations with Taxol are shown in Table 4. The MTD of CI-994 was 60 mg/kg/day. This dose produced a 12.5% complete tumor response rate and no partial tumor responses. The tumor growth delay for the tumors that did not completely respond to CI-994 was 14.9 days. This delay represents a net tumor cell kill of −0.18 log₁₀. None of the mice were tumor free when the study ended 74 days after the last CI-994 treatment. CI-994 at 30 mg/kg/day (50% its MTD) did not produce any complete or partial tumor responses. The tumor growth delay produced by this dose was 3.2 days, which represents a net tumor cell kill of −0.7 log₁₀. The MTD of Taxol was 20 mg/kg/day. This dose produced not complete or partial tumor responses. The tumor growth delay produced by Taxol at its MTD was only 2.5 days. This represents a net tumor cell kill of −0.07 log₁₀.

CI-994 could not be given at its MTD with Taxol at its MTD because of unacceptable lethality and weight loss. However, CI-994 could be given at 50% its MTD with Taxol at its MTD. This combination produced complete and partial tumor response rates of 37.5% to 25%, respectively. The tumor growth delay for the tumors that did not completely respond to this combination was 17.7 days, which represents net tumor cell kills of −0.06 log₁₀. There were 12.5% of the mice tumor free when the study ended 74 days after the last CI-994 treatment.

Better antitumor activity was seen when CI-994 was given at its MTD with Taxol at 50% its MTD. This combination produced a 100% complete tumor response rate and 100% of the mice were still tumor free when the study ended 74 days after the last CI-994 treatment.

These results indicate the antitumor activity is greater than additive when CI-994 is given at its MTD the same time as Taxol at 50% its MTD.

TABLE 4
Antitumor Effect of the Combinations of CI-9-94 and Taxol in the LC-12 Mouse Lung Tumor Model:
Effect of Simultaneous Treatment Against Advanced Stage Tumor

| CI-994 | | Taxol | | Toxic | % Weight | Antitumor Effect | | | | |
|---|---|---|---|---|---|---|---|---|---|---|
| Dose^{a} | Schedule | Dose^{a} | Schedule | Deaths | Change^{b} | CR^{c} | PR^{d} | % Tumor Free^{e} | T-C^{f} (+) | Net Log₁₀ Kill^{g} |
| — | — | — | — | 0/8 | +15.5 | 0/8 | 0/8 | 0/8 | 0.0 | 0.0 |
| 7.5 | 11-15, 18-22, 25-29 | — | — | 0/8 | +10.6 | 0/8 | 0/8 | 0/8 | 2.0 | −0.75 |
| 15.0 | | — | — | 0/8 | +3.4 | 0/8 | 0/8 | 0/8 | 0.5 | −0.82 |
| 30.0 | | — | — | 0/8 | −1.4 | 0/8 | 0/8 | 0/8 | 3.2 | −0.70 |
| 60.0 | | — | — | 0/8 | −4.4 | 1/8 | 0/8 | 0/8 | 14.9 | −0.18 |
| — | — | 15 | 11-15 | 0/8 | −3.9 | 0/8 | 0/8 | 0/8 | 0.0 | −0.22 |
| — | — | 20 | | 0/8 | −8.2 | 0/8 | 0/8 | 0/8 | 2.5 | −0.07 |
| 7.5 | 11-15, 18-22, 25-29 | 15 | 11-15 | 0/8 | −3.2 | 0/8 | 0/8 | 0/8 | 2.2 (2.0) | −0.74 |
| 15.0 | | | | 0/7 | −13.1 | 1/8 | 0/8 | 1/8 | 8.3 (0.5) | −0.47 |
| 30.0 | | | | 0/8 | −14.1 | 1/8 | 5/8 | 1/8 | 13.9 (3.2) | −0.23 |
| 60.0 | | | | 0/8 | −17.1 | 8/8 | — | 8/8 | >103.0 (14.9) | >3.72 |
| 7.5 | 11-15, 18-22, 25-29 | 20 | 11-15 | 0/8 | −10.1 | 0/8 | 0/8 | 0/8 | 2.5 (4.5) | −0.73 |
| 15.0 | | | | 1/8 | −11.6 | 1/8 | 2/8 | 1/8 | 8.9 (3.0) | −0.45 |
| 30.0 | | | | 0/8 | −13.9 | 3/8 | 2/8 | 1/8 | 17.7 (5.7) | −0.06 |
| 60.0 | | | | 3/8 | −20.0 | Toxic | Toxic | Toxic | Toxic | Toxic |

^{a}Doses are in mg/kg/day.
^{b}A weight loss is the maximum seen during treatment; a weight gain is the weight seen at the end of treatment.
^{c}Complete response (CR) is defined as the fraction of tumors that had a 100% decrease in tumor mass during the study.
^{d}Partial response (PR) is defined as the fraction of tumors that had at least a 50% decrease in measurable tumor mass during the study.
^{e}The percent tumor free represent the mice that had an undetectable tumor when the study ended on Day 103.
^{f}The difference in days for the treated and control tumors to reach 750 mg. The value in parenthesis represents the calculated T-C value for an additive antitumor effect.
^{g}Net log₁₀ tumor cell kill was calculated from the T-C value as described in Materials and Methods.

### EXAMPLE 2

The combination of CI-994 plus paclitaxel was evaluated in mouse colon carcinoma cells (recombinant 26:10 cells), and the data was analyzed according to the Chou and Talalay program which established both combinations to be synergistic.

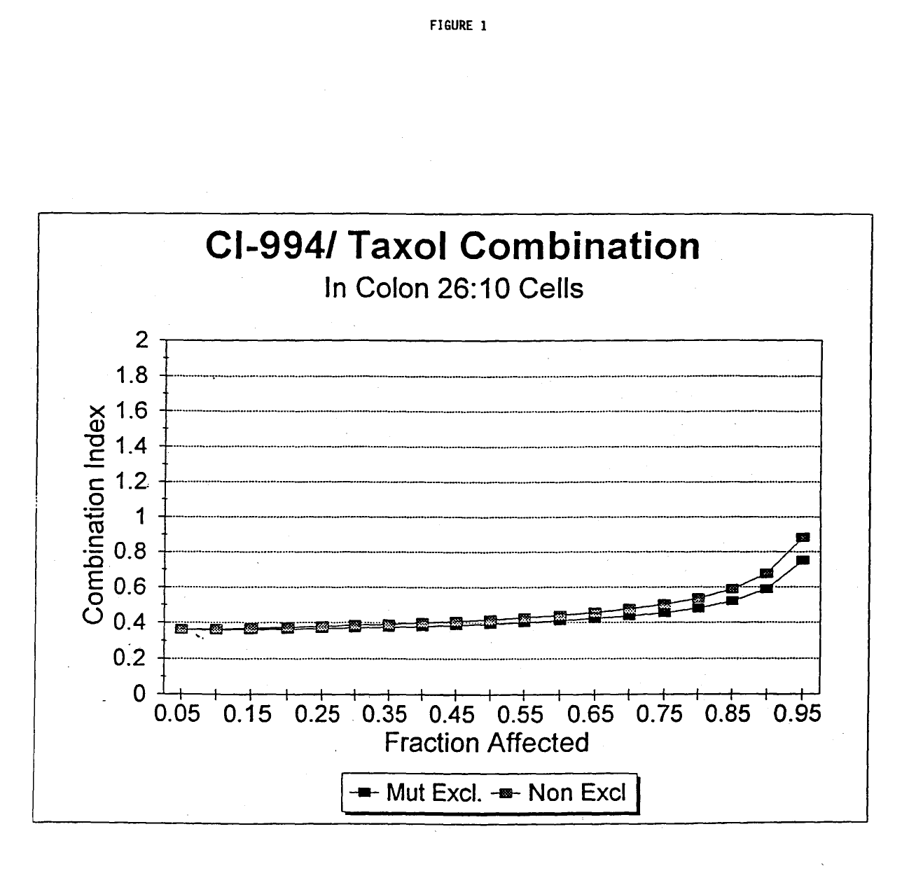

Mouse colon carcinoma cells were seeded into 96-well culture plates in RPMI 1640 culture media supplemented with 20% fetal calf serum and 10 μg/mL of insulin. Various concentrations of CI-994 and paclitaxel were added together 24 hours after cells were initially seeded into the culture plates and allowed to attach. The effect of CI-994 and Taxol alone and in combination on colon carcinoma proliferation was determined after 96 hours of incubation at 37° C. using the SRB assay (Skehan P., Stoneng R, Scudiero D, et al. New calorimetric cytotoxicity assay for anticancer-drug screening. J. Natl. Cancer Inst., 1990;82:1107-1112). The combination chemotherapy data was analyzed using the Biosoft program, “Dose Effect Analysis with Microcomputers for IBM PC,” which is a standard program for quantifying synergism, additivism, and antagonism among anticancer agents, and is based on the median-effect principle of mass-action law using an enzyme kinetic system model described by Chou and Talalay. Plots of fraction affected (Fa) versus combination index (CI) are called Fa-CI plots. These plots indicate synergism, additivity, or antagonism of 2 drugs at various effect levels in a mixture that is serially diluted. If several mixture are made, it is possible to estimate the optimal combination ratio for maximal synergy. Different effect levels usually give different degrees of synergism, additivism, or antagonism. CI values <1 indicate synergism; CI values >1 indicate antagonism and values that hover around 1 as a straight line indicate additivity. FIG. 1 shows representative Fa-CI plots for CI-994 plus Taxol. In the plots, CI values over the entire Fa range are less than one, indicating synergy for the drug combinations.

### EXAMPLE 3

The general procedure described in Example 1 was followed to evaluate the antitumor activity of CI-994 in combination with carboplatin.

CI-994 was given orally for three treatment cycles. Each cycle consisted of daily treatments for 5 days followed by 2 days rest (15 days total treatment). With this schedule, the MTD was 60 mg/kg. This dose produced 50% complete responses and no partial responses. Mice whose tumor completely responded were tumor free when the study ended on Day 71. For the tumors that didn't completely respond, a tumor growth delay of 19.4 days was produced by CI-994 at its MTD. This growth delay represents a net tumor cell kill of 0.1 log₁₀. When CI-994 was given at 30 mg/kg, there were 20% complete responses, 10% partial responses, and a tumor growth delay of 8.2 days. Little antitumor activity was seen with lower CI-994 doses. Carboplatin (Paraplatin®, Bristol-Myers Squibb Co., Princeton, N.J.) was given ip, once a day, for 5 days. At its MTD of 20 mg/kg, carboplatin produced 20% complete responses, 10% partial responses, and a tumor growth delay of only 5.9 days. Ten percent complete responses, no partial responses, and a tumor growth delay of 8.3 days were produced with carboplatin at 75% of its MTD (15 mg/kg). Carboplatin was given with CI-994 during the first CI-994 treatment course. When given in this manner, CI-994 could be given at only 25% of its MTD with carboplatin at its MTD without increased lethality. This dose combination produced 70% complete and 10% partial tumor responses. Sixty percent of the mice were tumor free at the end of the study on Day 71. The tumor growth delay for the tumors that didn't completely respond, or regrew, was 20.8 days. This represents a greater than additive antitumor effect. When CI-994 was given at its MTD with carboplatin at 75% of its MTD, there were 100% complete tumor responses. One hundred percent of the surviving mice were tumor free when the study ended. This represents a net tumor cell kill of at least 3.8 log₁₀, which is clearly a greater than additive antitumor effect. There was one death in this group, but the cause of this death is unknown.

The results of the foregoing study are presented in Table 5. The results establish that CI-994 can be given with carboplatin to produce a greater than additive antitumor effect without a significant increase in toxicity. This results in a therapeutic effect that is superior to the effects produced by either drug alone at their MTDs.

TABLE 5
Antitumor Effect of Treatment With CI-994 and Carboplatin Against Advanced Stage LC-12 Mouse Lung Carcinoma

| | | | | | | Antitumor Effect | | | | |
|---|---|---|---|---|---|---|---|---|---|---|
| CI-994 | | Carboplatin | | No. of | % Weight | | | T-C^{e} | Net Log₁₀ | No. Tumor |
| Dose^{a} | Schedule | Dose^{a} | Schedule | Deaths | Change^{b} | CR^{c} | PR^{d} | (additive) | Kill^{f} | Free^{g} |
| 0 | — | 0 | — | 0/10 | +10.4 | 0/10 | 0/10 | — | — | 0/10 |
| 7.5 | Days 8-12,15-19, 22-26 | 0 | — | 0/10 | +4.4 | 2/10 | 0/10 | 1.0 | −1.2 | 2/10 |
| 15.0 | | 0 | — | 0/10 | +4.9 | 1/10 | 0/10 | 0.9 | −1.3 | 1/10 |
| 30.0 | | 0 | — | 0/10 | +2.0 | 2/10 | 1/10 | 8.2 | −0.7 | 2/10 |
| 60.0 | | 0 | — | 0/10 | −1.5 | 5/10 | 0/10 | 19.4 | +0.1 | 5/10 |
| 0 | — | 15 | Days 8-12 | 1/10^{h} | −2.0 | 1/10 | 0/10 | 8.3 | +0.3 | 0/9 |
| 0 | — | 20 | | 0/10 | −5.5 | 2/10 | 1/10 | 5.9 | +0.1 | 2/10 |
| 7.5 | Days 8-12,15-19, 22-26 | 15 | Days 8-12 | 0/10 | −2.0 | 0/10 | 0/10 | 7.7 (9.3) | −0.8 | 0/10 |
| 15.0 | | 15 | | 0/10 | −5.6 | 4/10 | 1/10 | 15.0 (9.2) | −0.2 | 2/10 |
| 30.0 | | 15 | | 0/10 | −9.6 | 8/10 | 1/10 | 15.9 (16.5) | −0.2 | 8/10 |
| 60.0 | | 15 | | 1/10^{h} | −16.6 | 10/10 | 1/10 | >71.0 (27.7) | >+3.8 | 9/9 |
| 7.5 | Days 8-12,15-19, 22-26 | 20 | Days 8-12 | 0/10 | −6.4 | 0/10 | 1/10 | 12.7 (6.9) | −0.4 | 0/10 |
| 15.0 | | 20 | | 0/10 | −7.0 | 7/10 | 1/10 | 20.8 (6.8) | +0.2 | 6/10 |
| 30.0 | | 20 | | 3/10^{h} | −11.3 | 8/10 | 1/10 | 53.9 (14.1) | +2.6 | 6/7 |
| 60.0 | | 20 | | 3/10^{h} | −14.2 | 10/10 | 0/10 | >71.0 (25.3) | >+3.8 | 7/7 |

^{a}Doses are in mg/kg/day. Treatments were started when the tumor weights were between 189 and 245 mg (median = 245 mg).
^{b}A weight loss is the maximum seen during treatment; a weight gain is the weight seen at the end of treatment.
^{c}A complete response represents a tumor whose mass decreased by 100% during the study.
^{d}A partial response represents a tumor whose mass decreased by at lease 50% during the study.
^{e}T-C is the difference in days for the treated and control tumors to reach 750 mg. The values in parenthesis represent the T-C values for an additive antitumor effect.
^{f}Net log₁₀ tumor cell kill was calculated from the T-C value as described in Materials and Methods.
^{g}The number tumor free represents the mice that had an undetectable tumor when the study ended on Day 71.
^{h}Deaths were delayed and primarily occurred 1 to 2 weeks after the last treatment with carboplatin.

### EXAMPLE 4

The general procedure described in Example 1 was followed to evaluate the antitumor activity of CI-994 in combination with both paclitaxel and with carboplatin. Each agent was also evaluated alone, and treatment groups were compared to untreated controls. The MTD of CI-994 was 60 mg/kg/day. This dose produced a 30 percent complete response rate and no partial tumor responses. The mice whose tumors completely responded were still tumor free 40 days after the last treatment with CI-994. The tumor growth delay for the tumors that did not completely respond to CI-994 was 16.2 days. Paclitaxel and carboplatin were both given alone at their MTD's of 15 and 30 mg/kg, respectively. There were no complete or partial tumor responses and only a 3.7 day tumor growth delay with paclitaxel at its MTD. At its MTD, carboplatin did not produce any complete or partial tumor responses. The tumor growth delay for this dose of carboplatin was 6.9 days. The results of this study are presented in Table 6.

CI-994 could not be given at its MTD with paclitaxel and carboplatin at its MTD because of unacceptable deaths. When CI-994 was given at 50% of its MTD with these drugs at their MTD's, there was one death. However, this dose combination produced 80 percent tumor responses and the mice were still tumor free 40 days after the last CI-994 treatment. The tumor growth delay for the tumors that did not completely respond was greater than 51 days. These results indicate the antitumor effect is synergistic when tumor bearing mice are treated with the combination of CI-994, paclitaxel and carboplatin.

TABLE 6
Antitumor Effect of the Combination of CI-994, Paclitaxel and Carboplatin

| CI-994 | | Paclitaxel | | Carboplatin | | Nonspecific | % Weight | | | | Net | No. Tumor |
|---|---|---|---|---|---|---|---|---|---|---|---|---|
| Dose^{a} | Schedule | Dose^{a} | Schedule | Dose^{a} | Schedule | Deaths | Change^{b} | CR^{c} | PR^{d} | T-C | log₁₀ Kill^{f} | Free^{g} |
| 0 | none | 0 | none | 0 | none | | | | | | | |
| 15 | 11-15,18-22,25-29 | 0 | none | 0 | none | 0/10 | +4.0 | 0 | 0 | 4.6 | −1.3 | 0 |
| 30 | \| | 0 | \| | 0 | \| | 0/10 | +1.0 | 3 | 0 | 13.4 | −0.4 | 2 |
| 60 | \| | 0 | \| | 0 | \| | 0/9 | −5.6 | 3 | 0 | 16.2 | −0.2 | 3 |
| 0 | none | 15 | 11-15 | 0 | none | 0/10 | −3.0 | 0 | 0 | 3.7 | 0.0 | 0 |
| 0 | none | 0 | none | 30 | 11-15 | 0/10 | −3.9 | 0 | 0 | 6.9 | 0.3 | 0 |
| 7.5 | 11-15,18-22,25-29 | 10 | 11-15 | 30 | 18-22 | 0/9 | −2.0 | 3 | 0 | 15.5 | −0.2 | 1 |
| 15 | \| | \| | \| | \| | \| | 0/10 | −2.0 | 3 | 0 | 14.8 | −0.3 | 3 |
| 30 | \| | \| | \| | \| | \| | 1/10 | −7.7 | 8 | 0 | >51 | >2.0 | 8 |
| 60 | \| | \| | \| | \| | \| | 8/10 | Toxic | | | | | |
| 7.5 | 11-15,18-22,25-29 | 15 | 11-15 | 30 | 18-22 | 0/10 | −3.5 | 3 | 0 | 6.2 | −1.1 | 3 |
| 15 | \| | \| | \| | \| | \| | 0/10 | −5.9 | 5 | 0 | 9.3 | −0.8 | 5 |
| 30 | \| | \| | \| | \| | \| | 1/10 | −12.3 | 8 | 0 | >51 | >2.0 | 8 |

| | | | | | | | |
|---|---|---|---|---|---|---|---|
| 60 | \| | \| | \| | \| | \| | 10/10 | Toxic |

^{a}Doses are in mg/kg/day. Treatments were started when the tumor weights were between 120 and 269 mg.
^{b}A weight loss is the maximum seen during treatment; a weight gain is for the weight seen at the end of treatment.
^{c}Complete response represents a tumor that decreased in mass by 100% during the study.
^{d}Partial response represents a tumor that decreased in mass by at least 50% during the study.
^{e}The difference in days for the treated and control tumors to reach 750 mg.
^{f}Net log₁₀ tumor cell kill was calculated from the T-C value as described by in Materials and Methods.
^{g}The percent tumor free represents the mice that had an undetectable tumor 40 days after the last treatment.

### EXAMPLE 5

Clinical Evaluation of 2-Component Combination Therapy

This is a multicenter, open-label Phase 1 study of CI-994 given in combination with paclitaxel to patients with advanced solid tumors.

The objectives of this study are to determine the (1) MTD, (2) recommended Phase 2 dose, (3) pharmacokinetics, (4) safety profile, and (5) to observe for antitumor activity of CI-994 when given in combination with paclitaxel to patients with advanced solid tumors. The primary efficacy endpoint is the attainment of either a PR or a CR. Secondary endpoints include time to PR or CR, duration of PR or CR, and survival.

Paclitaxel is administered as an intravenous infusion at 3-week intervals during the treatment course, using an initial dose of 135 mg/m². CI-994 is administered orally as a daily dose for 21 days of a 28-day course, beginning on Day 1. Patients may receive subsequent courses of treatment based on individual tolerance and response to therapy. Patients whose disease does not respond or who develop intolerable adverse events are discontinued from study treatment.

The initial dose level of CI-994 is 4 mg/m². A minimum of three patients will be treated at each dose level. Dose levels are increased by 2 mg/m² until the MTD is reached. Ten additional patients are to be treated at the dose level recommended for Phase 2 studies, which is expected to be the MTD or one dose level below the MTD.

Once a patient begins study treatment, the addition of other cancer treatment will confound the assessment of safety and efficacy and therefore is not allowed. This restriction precludes the addition of systemic cytotoxic, hormonal, immunologic, or other biologic agents while the patient is in the treatment phase of this protocol. Patients who require palliative radiotherapy while on study are generally considered to have progressive disease and, unless compelling information exists to the contrary, are to be discontinued from study medication. Patients who develop new brain metastases while on study may have treatment interrupted to receive a course of cranial irradiation, then be restarted on study medication after a recovery period of at least 1 week.

Antiemetics may be used at the investigator's discretion for prevention and/or treatment of nausea or vomiting. Every effort should be made to ensure nausea and vomiting is controlled, as these conditions may preclude a patient from taking or absorbing the oral doses of CI-994. This issue is particularly relevant on Days 1, 8, and 15, when gemcitabine is also administered. Because CI-994 caused sedation and somnolence at higher doses in the Phase 1 program, antiemetics that are least likely to cause these side effects should be used.

Colony-stimulating factors may be used at the investigator's discretion to treat episodes of severe myelosuppression that are complicated by infection, but should otherwise not be used to support low blood counts or to maintain dose intensity.

If criteria are met for a CR, administer 2 additional courses of treatment beyond confirmation of the CR and then completely reassess the patient's disease state. If the patient is considered to be clinically free of disease at that time, discontinue the gemcitabine and continue to administer CI-994 for 3 additional months, using the same dose and schedule (3 weeks on/I week off). At that time, again completely reassess the patient's disease state. If the patient is still in CR, the investigator must evaluate the risks and potential benefits of continuing CI-994 treatment.

Treatment Courses

A treatment course consists of Taxol given intravenously on Day 1 of a 28-day course plus CI-994 administered daily orally, beginning on Day 1, for 21 days of a 28-day course. Courses are to be repeated on Day 29 if there has been adequate recovery from adverse events and myelosuppression, defined as nonhematologic parameters of Grade ≦1, platelet count ≧100,000 μL, and absolute neutrophil count ≧1500 μL. Subsequent courses may be delayed by weekly intervals up to 3 weeks. If recovery has not occurred by Day 50, the patient is to be discontinued from study medication.

Taxol Dosing

The initial dose of Taxol in each course is 135 mg/m², given as a 3-hour intravenous infusion. Dose adjustments may be required during a treatment course. Follow the manufacturer's recommendations for information regarding preparation and administration.

CI-994 Dose Levels

CI-994 doses are calculated based on body surface area (BSA) and must then be rounded to the closest available capsule strength. CI-994 is available in capsule strengths of 2.5, 5, and 25 mg. Doses may be taken without regard to meals.

The initial CI-994 dose level is 4 mg/m². Subsequent dose levels will be increased (or decreased if necessary) by a fixed increment of 2 mg/m² until the MTD is determined. Individual patients may not receive dose escalations of gemcitabine or CI-994 in subsequent courses. Patients may receive a lower dose of CI-994 in a subsequent course if dose-limiting toxicities were experienced.

Three new patients will be assessed at each new dose level. The minimum time that these patients must be followed is 4 weeks before a new dose level may be opened (unless treatment was interrupted earlier and the patient is recovering from adverse events). If none of these three patients experience a dose-limiting toxicity, the next higher dose level will be opened. If one patient develops a dose-limiting toxicity, three more patients will be enrolled at that dose level. If ≧2 of 6 patients experience a dose-limiting toxicity at the same level, that dose level will be considered the MTD.

An assessable patient is defined as one who received 3 weekly doses of gemcitabine plus at least 80% of the CI-994 doses (≧17 doses), or a patient whose treatment course was discontinued early or was noncompliant (<17 doses) due to treatment-related adverse events. A patient who took fewer than 17 doses of CI-994 or did not complete the treatment course because of nontreatment-related reasons (e.g., missed appointments, ran out of CI-994 supplies, developed a coexisting medical condition that rendered the patient unable to swallow capsules, developed rapidly progressing disease) is not considered to be an assessable patient for the tolerability of that dose level.

Patients should be encouraged to take their CI-994 dose at approximately the same time each day. However, a variance of up to 12 hours either way is allowed for any given dose, rather than miss a day's dose. If a patient misses a day's dose entirely, they must be instructed not to ‘make it up’ the next day. If a patient vomits anytime after taking a dose of CI-994, they must be instructed not to ‘make it up’, but to resume subsequent doses the next day as prescribed.

On Days 1, 8, and 15, the CI-994 dose should be given 2 hours before the gemcitabine dose, to ensure maximal absorption in the event the patient develops vomiting following the gemcitabine dose.

Once the MTD is determined, 10 additional patients will be treated at the Phase 2 dose level, which is expected to be the MTD or one dose level below the MTD.

Dose Adjustments During a Course

Continuation of Taxol and CI-994 during a course is dependent on patient tolerance and hematologic parameters. Reduced doses of gemcitabine may be required on Days 8 and 15, as recommended by the manufacturer and shown in the table below. The dose of CI-994 is not to be increased or decreased during a treatment course, although early termination may be required as described below. If both study medications must be stopped before a course is completed, do not complete that course, but instead follow the patient for recovery, then start another course using a reduced dose of CI-994.

The decision to discontinue CI-994 dosing during a course is based on adverse events or hematology results at any time. Example: A patient has a platelet count on Day 11 of 45,000 μL. Instruct the patient to stop taking CI-994 capsules (and to return all study medication containers to the site). Obtain another CBC on Day 15. If the platelet count on Day 15 is 50,000 to 99,000 μL, administer 75% of the calculated Taxol dose but do not reinstitute CI-994 dosing. If the platelet count on Day 15 remains below 50,000/μL, do not retreat with gemcitabine. Consider this course to be terminated and follow the patient for recovery. In either case, the patient may receive a subsequent treatment course using the same initial dose of Taxol and a CI-994 dose that has been reduced by 2 mg/m².

Drug Formulation and Stability

Taxol is to be obtained by the site from commercial sources. Follow the manufacturer's recommendation for preparation, administration, stability, and storage conditions.

CI-994 is formulated in identically appearing gelatin capsules containing 2.5, 5, or 25 mg of study medication, plus inactive ingredients of lactose, cornstarch, and talc or polyethylene glycol 6000. Store at controlled room temperature.

### EXAMPLE 6

Clinical Evaluation of 3-Component Combination Therapy

This is a clinical study of oral CI-994 in combination with paclitaxel and carboplatin in the treatment of patients with advanced solid tumors. Patients to be treated will have advanced solid tumors and will not have received more than two prior chemotherapy regimens, and for whom paclitaxel and carboplatin are reasonable treatment options. Primary efficacy parameters will include response to treatment classified as complete remission (CR), partial remission (PR), stable disease (SD), or progressive disease (PD).

A treatment course consists of paclitaxel and carboplatin given intravenously on Day 1 of a 21-day course plus CI-994 administered daily orally, beginning on Day 1, for 7 or 14 days, depending on the dose level, of a 21-day course. Courses are to be repeated on Day 22 if there has been adequate recovery from adverse events and myelosuppression. Subsequent courses may be delayed by weekly intervals up to 3 weeks. If recovery has not occurred by Day 43, the patient is to be discontinued from study medication.

Paclitaxel is to be obtained by the site from commercial sources. Follow the manufacturer's recommendations for information regarding preparation, administration, storage, and stability. In particular, note the requirement to avoid PVC-containing infusion sets and bags and the recommendation to use an in-line filter.

Patients must be premedicated prior to receiving each dose of paclitaxel. The pretreatment regimen currently recommended by the manufacturer consists of dexamethasone 20 mg PO administered approximately 12 and 6 hours before paclitaxel, diphenhydramine 50 mg IV 30 to 60 minutes prior to paclitaxel, and cimetidine (300 mg) or ranitidine (50 mg) IV 30 to 60 minutes. Vital signs should be monitored during paclitaxel infusions in accordance with institutional policy.

Despite premedication, anaphylactic reactions have occurred with paclitaxel infusions. Adequate medical supervision must be present and appropriate intervention must be readily available to diagnose and treat hypersensitivity reactions.

Carboplatin is to be obtained by the site from commercial sources. Follow the manufacturer's recommendations for information regarding preparation, administration, storage, and stability. In particular, note the requirement to avoid needles or IV sets that have aluminum components.

Anaphylactic reactions have occurred with carboplatin injections. Adequate medical supervision must be present and appropriate intervention must be readily available to diagnose and treat hypersensitivity reactions.

Carboplatin is given as a 30-minute IV infusion immediately following the completion of the paclitaxel infusion. The carboplatin dose (in mg) to use in each course is calculated to produce an area under the concentration time curve (AUC) of 6 using the Calvert formula, substituting the creatinine clearance as determined by the Cockcroft-Gault formula for the glomerular filtration rate (GFR).

Dose (in mg)=AUC of 6 (GFR+25)

$$GFR = \frac{[ ( 140 - age ) \times weight in kg )}{( 72 \times serum creatinine ) ]} \times 0.85 for females$$

EXAMPLE: Patient characteristics are female, 63 years old, serum creatinine=1.1 mg/dL, weight=66 kg.

$$GFR = \frac{( 140 - 63 ) \times 66}{72 \times 1.1} \times 0.85 = 54.5$$

Carboplatin dose in mg=6 (54.5+25)=477 mg (NOT mg/m²)

CI-994 doses are calculated based on body surface area (BSA) and must then be rounded to the closest available capsule strength. CI-994 is available in capsule strengths of 2.5, 5, 10, and 25 mg. Doses may be taken without regard to meals.

Patients should be encouraged to take their CI-994 dose at approximately the same time each day. However, a variance of up to 12 hours either way is allowed for any given dose, rather than miss a day's dose. If a patient misses a day's dose entirely, they must be instructed not to ‘make it up’ the next day. If a patient vomits anytime after taking a dose of CI-994, they must be instructed not to ‘make it up’, but to resume subsequent doses the next day as prescribed.

A Study Medication Diary is to be completed during each treatment course [date, dose taken or not, vomited or not within 2 hours after taking dose]. On Day 1 of each course, the CI-994 dose should be given 2 hours before the paclitaxel dose, to ensure maximal absorption in the event the patient develops vomiting following the paclitaxel dose.

### Dose Levels and Number of Patients

The following table describes the anticipated dose levels. It is designed to reach the ‘standard’ dosing of paclitaxel (225 mg/m²) and carboplatin (AUC=6).

| | | | |
|---|---|---|---|
| | Paclitaxel Dose (mg/m²) | Carboplatin AUC, | |
| Dose | infused over 3 hours on | infused over | |
| Level | Day 1 | 30 minutes on Day 1 | CI-994 Dose orally |

14-DAY SCHEDULE CI-994

| | | | |
|---|---|---|---|
| −2 | 175 | 6 | 2.5 mg fixed dose on |
| | | | Days 1-14 |
| −1 | 175 | 6 | 5 mg fixed dose on |
| | | | Days 1-14 |
| 1 | 175 | 6 | 4 mg/m²/day on Days 1-14 |
| 2 | 175 | 6 | 6 mg/m²/day on Days 1-14 |
| 3* | 200 | 6 | 4 mg/m²/day on Days 1-14 |
| 4* | 225 | 6 | 4 mg/m²/day on Days 1-14 |
| 5* | 225 | 6 | 6 mg/m²/day on Days 1-14 |

7-DAY SCHEDULE CI-994

| 6* | 200 | 6 | 4 mg/m²/day on Days 1-7 |
|---|---|---|---|
| 7* | 225 | 6 | 4 mg/m²/day on Days 1-7 |
| 8+ | 225 | 6 | 6 mg/m²/day on Days 1-7 |
| *After Dose Level 3, Dose Level 7 or higher will be opened sequentially in the event that the results of the ongoing patient experience indicated adequate tolerability of the preceding dose level. Dose Level 3 has met MTD criteria, and thus Dose Levels 4 and 5 will NOT be evaluated immediately following Dose Level 3. Dose Level 6 will NOT be | | | |
| #evaluated immediately following Dose Level 3, as experience at this dose level has been obtained via early patient discontinuation of CI-994 in Dose Level 3 (approximating Dose Level 6's abbreviated CI-994 dosing schedule). Dose Level 6 will be opened if Dose Level 7 meets the criteria for MTD. | | | |

+Should Dose Level 8 not meet the MTD, additional dose levels will be opened at increments of 2 mg/m²/day CI-994. Each of these Dose Levels would include paclitaxel and carboplatin as described for Dose Level 8.

The initial CI-994 dose level is 4 mg/m² which represents approximately 25% of the MTD when CI-994 was given as a single agent for 14 days in a Phase 1 study, and 50% of the daily dose when given as a single agent on a chronic daily basis in the ongoing Phase 2 program. See the section for a summary of the CI-994 clinical experience.

Three new patients will be assessed at each new dose level. The minimum time that these patients must be followed is 3 weeks before a new dose level may be opened (unless treatment was interrupted earlier and the patient is recovering from adverse events). If none of these 3 patients experience a dose-limiting toxicity of any kind, OR if 1 patient develops dose-limiting neutropenia (see criteria in dose-limiting toxicity section), the next higher dose level will be opened. If 1 patient develops a dose-limiting toxicity except neutropenia, OR if 2 patients develop dose-limiting neutropenia, 3 more patients will be enrolled at that dose level. If ≧2 of 6 patients experience a dose-limiting toxicity except neutropenia at the same level, OR if ≧3 of 6 patients develop dose-limiting neutropenia, that dose level will be considered the MTD.

| | | |
|---|---|---|
| Number of Patients | Number of Patients | |
| Meeting DLT Criteria, | Meeting Neutropenia | |
| Excluding Neutropenia | DLT Criterion | Action |

Initial Assessment of Dose Level (3 patients enrolled to dose level):

| | | | |
|---|---|---|---|
| 0/3 | OR | ≦1/3 | Dose escalate |
| 1/3 | OR | 2/3 | Enroll 3 |
| | | | additional patients |
| | | | at the same dose |
| | | | level |
| ≧2/3 | OR | 3/3 | MTD met |

Final Assessment of Dose Level (6 patients enrolled to dose level):

| | | | |
|---|---|---|---|
| ≦1/6 | OR | ≦2/6 | Dose escalate |
| 1/6 | AND | 2/6 | Dose escalate |
| ≧2/6 | OR | ≧3/6 | MTD met |

Once the MTD is determined, 6 additional patients will be treated at the Phase 2 dose level, which is expected to be the MTD or one dose level below the MTD.

To be considered an assessable patient for the safety of a given dose level, a patient must have received a full dose of paclitaxel and carboplatin plus at least 75% of the CI-994 regimen, or was discontinued early or was noncompliant due to treatment-related adverse events. A 75% compliance level is >11 doses on the 14-day schedule and >6 doses on the 7-day schedule. A patient who took fewer than 75% of the doses of CI-994 or did not complete the treatment course because of nontreatment-related reasons (e.g., missed appointments, misplaced their CI-994 supplies, developed a coexisting medical condition that rendered the patient unable to swallow capsules, developed rapidly progressing disease) is not considered to be an assessable patient for the tolerability of that dose level.

Dose Adjustments During a Course

Paclitaxel and carboplatin are both given only on Day 1 of each treatment course and as such are not subject to dose adjustments during a course. Continuation of CI-994 during a course is dependent on patient tolerance and hematologic parameters. The dose of CI-994 is not to be increased or decreased during a treatment course, although early termination may be required. Discontinue CI-994 dosing during a treatment course if any of the following conditions are met:

Platelet count is <40,000 μL;

Absolute neutrophil count is <500/μIL; and

Nonhematologic treatment-related adverse event is >Grade 3 (except alopecia or controllable nausea or vomiting).

Instruct the patient to stop taking CI-994 capsules and to return all study medication containers to the site. Follow the patient for recovery, then start another course using reduced doses of study medications as described below in the Dose Adjustments and Timing of the Next Treatment Course section.

Dose Adjustments and Timing of the Next Treatment Course

The dose adjustments for paclitaxel, carboplatin, and CI-994 for the next treatment course are based on the absolute neutrophil count nadir, the platelet count nadir, and nonhematologic toxicities experienced during the prior course. Dose increases of paclitaxel, carboplatin, or CI-994 are not permitted in subsequent courses, other than carboplatin dose increases that result from recalculation based on the patient's current serum creatinine value and weight.

| | Dose Adjustments for Next Treatment Course Within a Patient | | |
|---|---|---|---|
| Maximum Condition | % of Prior | % of Prior | Dose Level Adjustment for |
| From Prior Course | Paclitaxel Dose | Carboplatin Dose^{a} | CI-994 |
| Absolute Neutrophil | | | |
| Count Nadir | | | |
| ≧500/μl | 100 | 100 | No change |
| <500/μl | 75 | 75 | Decrease by 2 mg/m²/day^{b} |
| - OR - | | | |
| Platelet Count Nadir | | | |
| ≧40,000/μl | 100 | 100 | No change |
| <40,000/μl | 75 | 75 | Decrease by 2 mg/m²/day^{b} |
| - OR - | | | |
| Treatment-Related | | | |
| Nonhematologic Toxicity | | | |
| Grade 0 to 2 (other than CNS) | 100 | 100 | No change |
| Grade 2 CNS; | 75 | 75 | Decrease by 2 mg/m²/day^{b} |
| Grade 3 or 4 (exclude alopecia | | | |
| or | | | |
| Controllable nausea or | | | |
| vomiting) | | | |

^{a}If a dose reduction is required, recalculate the carboplatin dose to an AUC = 6, using the patient's most recent parameters, and then give 75% of this dose.
^{b}If the patient is receiving a total dose of 5 mg/day, the patient's dose may be decreased to 2.5 mg/day, if in the investigator's clinical judgment, the patient would benefit from further protocol treatment. This decision should be reached in conjunction with the sponsor based upon the patient's tolerance and response to therapy.

Delay the start of the next treatment course until all of the following conditions are met:

Absolute neutrophil count is ≧2000/μL;

Platelet count is ≧100,000/μL; and

Nonhematologic treatment-related toxicities have recovered to Grade 0 or 1, with the exception of alopecia.

If a patient undergoes multiple dose decreases in subsequent courses such that he/she no longer is receiving any amount of CI-994, or experiences a significant hypersensitivity reaction to paclitaxel or carboplatin that precludes rechallenge, that patient is to be discontinued from the study.

Dose-Limiting Toxicity

Any of the following conditions is considered to be a dose-limiting toxicity (DLT):

Platelet count nadir <25,000/μL;

Absolute neutrophil count <500/μL for 5 days or more, or was associated with an infection or fever;

Grade 2 treatment-related CNS toxicities such as sedation, somnolence, disorientation, confusion, or hallucinations lasting for ≧24 hours;

Grade 3 or 4 treatment-related nonhematologic toxicities (except alopecia of any grade, or controllable nausea or vomiting);

Failure to recover from adverse events (except alopecia) or hematologic toxicities by Day 43; and

A terminated or noncompliant treatment course (fewer than 11 doses of CI-994 taken on the 14-day schedule; fewer than 6 doses of CI-994 taken on the 7-day schedule) due to a treatment-related toxicity of any grade.

Maximum-Tolerated Dose

The MTD is that dose level which produces any DLT except dose-limiting neutropenia in ≧2 of 6 assessable patients, OR dose-limiting neutropenia in ≧3 of 6 assessable patients in their first treatment course.

Safe Handling

Paclitaxel, carboplatin, and CI-994 are cytotoxic agents that must be handled and administered with care. Inhalation of powder or contact with skin and mucous membranes, especially those of the eyes, must be avoided. Should accidental eye contact occur, copious irrigation with water should be instituted immediately, followed by prompt ophthalmologic consultation. Should accidental skin contact occur, the exposed area should be irrigated immediately with copious amounts of water for at least 15 minutes. As with other cytotoxic antineoplastic agents, appropriate precautions should be followed in accordance with OSHA Guidelines.

Drug Formulation and Stability

CI-994 is formulated in identically appearing gelatin capsules containing 2.5, 5, 10, or 25 mg of study medication, plus inactive ingredients of lactose, cornstarch, and talc or polyethylene glycol 6000. Store at controlled room temperature.

The foregoing data establish an unexpectedly favorable interaction between acetyldinaline in combination with either paclitaxel or carboplatin, and in combination with both paclitaxel and carboplatin. Accordingly, this invention provides a method of treating susceptible neoplasms comprising administering acetyldinaline in a regimen together with paclitaxel or carboplatin, or together with both paclitaxel and carboplatin. The combination generally will include each active ingredient packaged separately, thereby avoiding any interaction between the agents prior to administration. If desired, the individually packaged drugs can be placed in a single carton as a kit, thereby providing convenience to the attending physician or medical attendant. The susceptible neoplasms to be treated according to this invention include solid tumors, especially advanced solid tumors and nonsmall cell lung cancer, as well as renal cell cancer, pancreatic cancer, head and neck cancer, ovarian cancer, myeloma, prostate cancer, and breast cancer.

What is claimed is:

1. A combination of antineoplastic agents comprising an antitumor amount of acetyldinaline and an antitumor amount of paclitaxel and/or carboplatin.

2. A combination of claim 1 comprising acetyldinaline formulated as a capsule.

3. A combination of claim 2 comprising paclitaxel or a pharmaceutically acceptable salt thereof formulated as a sterile solution for intravenous infusion.

4. A combination of claim 3 comprising paclitaxel monohydrochloride.

5. A combination comprising acetyldinaline and paclitaxel.

6. A combination comprising acetyldinaline and carboplatin.

7. A combination comprising acetyldinaline, paclitaxel, and carboplatin.

8. A method of treating cancer comprising administering to an animal in need of treatment an antitumor amount of a combination of claims 1, 5, 6, and 7.

9. A method of claim 8 wherein the cancer treated is nonsmall cell lung cancer.

10. A method of claim 9 comprising administering acetyldinaline in combination with paclitaxel and/or carboplatin.

11. A method of claim 8 wherein the cancer treated is prostate cancer.

12. A method of claim 8 wherein the cancer treated is a locally advanced (nonresectable Stage II or Stage III) or metastatic (Stage IV) adenocarcinoma of the pancreas.

13. A kit comprising acetyldinaline in one compartment and paclitaxel in a second compartment.

14. A kit comprising acetyldinaline in one compartment and carboplatin in a second compartment.

15. A kit comprising acetyldinaline in one compartment, paclitaxel in a second compartment, and carboplatin in a third compartment.
